(19) United States
(12) Patent Application Publication
(10) Pub. No.: US 2008/0248871 A1
(43) Pub. Date: Oct. 9, 2008

(54) INTERFACE DEVICE

(76) Inventors: Anthony SZTURM, Winnipeg (CA); Christopher OTTO, Winnipeg (CA)

Correspondence Address: Muncy, Geissler, Olds & Lowe, PLLC, P.O. BOX 1364, FAIRFAX, VA, 22038-1364, US

(21) Appl. No.: 12/048,970

(22) Filed: Mar. 14, 2008

Related U.S. Application Data
(60) Provisional application No. 60/895,138, filed on Mar. 15, 2007.

(51) Int. Cl. A61B 5/103; A63F 9/24

(52) U.S. Cl. 463/36; 600/587

(57) ABSTRACT

The present invention relates generally relates to an interface device for operative connection between a computer and a one-dimensional, two-dimensional or three-dimensional motion input system and/or tactile force sensing input system. The interface device includes a first interface for operative connection to the input system and a second interface for operative connection to the computer; and a processor operatively connected to the first and second interfaces for receiving input signals from the motion tracking/tactile force input system. The device, or computer, configures the input signals to output signals for input to the computer for controlling the computer. The invention is particularly beneficial in a therapy/rehabilitation setting wherein people or patients can participate in almost any video game by configuring the input system to the video game through the interface device.

### RELATED APPLICATION

This application claims the benefit of priority from U.S. application 60/895,138 filed Mar. 15, 2007.

### FIELD OF THE INVENTION

The present invention relates generally relates to an interface device for operative connection between a computer and a one-dimensional, two-dimensional or three-dimensional motion input system and/or tactile force sensing input system. The interface device includes a first interface for operative connection to the input system and a second interface for operative connection to the computer; and a processor operatively connected to the first and second interfaces for receiving input signals from the motion tracking/tactile force input system. The device, or computer, configures the input signals to output signals for input to the computer for controlling the computer. The invention is particularly beneficial in a therapy/rehabilitation setting wherein people or patients can participate in almost any video game by configuring the input system to the video game through the interface device.

### BACKGROUND OF THE INVENTION

Rehabilitation science focuses on human functioning in environments critical and purposeful to individuals. The focus of rehabilitation is on people's ability to function within their environment and the related influences of pathophysiological, personal, societal and cultural variables. The diversity and heterogeneity of these populations with disabilities and handicaps require that treatments be flexible and accommodate individual differences.

For example, patients who have suffered brain injuries or have other neurological disorders will have varying levels of disabilities in the mobility of their extremities, particularly the mobility of their hands and fingers. Such disabilities may prevent the patient from performing various movements with their hands and fingers such as grasping or gripping, or simply controlled motion of their hands.

As part of the rehabilitation treatments for such disabilities, patients are often asked to perform repetitive tasks such as repeatedly gripping or squeezing an object or simply moving an object from one location to another in a controlled or smooth manner. While in a motivated patient, such treatments may be effective, in many cases, patients quickly lose interest in performing such repetitive tasks and, as a result, the treatment program is not fully completed.

As a result, there has been a need for treatment systems that provide the patient with motivation to continue treatment regimes by providing auditory or visual feedback to encourage them to continue. One such methodology to provide this level of feedback is to enable patients to play computer games as computer games can be entertaining, provide audio and visual feedback and provide a quantifiable result. However, computer games and computer systems are not set up to enable a wide range of physical movements as the input to the computer game. As a result, computer games leave only provided a very limited amount of rehabilitation value. Thus, there has been a need for a system wherein the input to the computer game is not provided by a standard input device such as a joystick, mouse or keypad but is provided by an input system that forces the patient to perform the desired therapeutic movement to operate the computer game.

A review of the prior art indicates that such a system has not been previously provided. For example, U.S. Pat. No. 6,413,190 (issued Jul. 2, 2002) discloses a rehabilitation apparatus and method that converts sensed muscle contraction or body movements into computer inputs, whereby therapy can be provided by requiring specific physical movement to accomplish computer game activities. This patent requires body sensors to be configured to a patient which detect muscle movement.

U.S. Patent Application 2004/0193413 (published Sep. 30, 2004) discloses a system for controlling a computer using hand gestures as alternatives to mouse operations for increasing computer accessibility to the disabled. In detecting hand movement, this system utilizes video cameras to recognize gestures of a user.

U.S. Pat. No. 5,139,261 (issued Aug. 18, 1992) discloses interfacing a video game device with exercise equipment in order to require pre-selected degrees of difficulty for operating a video game.

Other documents include U.S. Pat. No. 5,883,616 (issued Mar. 16, 1999) which discloses a computer input device utilizing an optical signal transmitter for severely disabled persons and U.S. Patent Application 2005/0041016 (published Feb. 24, 2005) which discloses an interface for users and control devices which can assist the disabled. U.S. Pat. No. 6,452,585 (issued Sep. 17, 2002), U.S. Pat. No. 5,990,865 (issued Nov. 23, 1999), U.S. Pat. No. 5,982,352 (issued Nov. 9, 1999), U.S. Pat. No. 5,288,078 (issued Feb. 22, 1994) and U.S. patent application Ser. No. 5,982,352 (published Mar. 24, 2005) each disclose various control systems and sensors that are alternatives to joysticks or a computer mouse.

### SUMMARY OF THE INVENTION

It is an object of the present invention to provide an effective interface between an input system and a computer to enable users to provide effective input to a computer through other input systems than those normally used with computers.

In accordance with a first embodiment, there is provided an interface device for operative connection between a computer and an input system comprising: a first interface for operative connection to the input system and a second interface for operative connection to the computer; and a processor operatively connected to the first and second interfaces for receiving input signals from the input system and configuring the input signals to output signals for input to the computer for controlling the computer.

In various embodiments, the processor includes means for dynamically selecting any one of or a combination of keyboard, mouse and joystick outputs, means for testing communication between the interface device and the computer, means for receiving input from a user to adjust the linear and angular motion or magnitudes of pressure of a sensor of the input system to a defined range of motion for input to the computer, means for testing input and output signals for simulating output signals to verify compatibility with the computer's software, and/or means for providing output signals to a USB port.

In still further embodiments, the interface device includes a keyboard input interface operatively connected to the processor for providing keyboard input to the processor and/or a display operatively connected to the processor for displaying graphic output to the user.

In another aspect, the invention provides a method of providing video gaming functionality to patients having compromised motion comprising the steps of: operatively connecting the interface device between an input system and computer; operatively connecting an exercise object to a motion sensor of the input system, and, configuring the interface device such that movement of the sensor results in appropriate input to a video game on the computer.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 is a schematic overview of an interface device connected to a computer and input system in accordance with the invention;

FIG. 2 is a schematic diagram of an interface device in accordance with one embodiment of the invention;

FIG. 3 is a schematic diagram of an interface device in accordance with one embodiment of the invention wherein the interface device is used as a secondary interface device;

FIG. 4 is a schematic diagram of the operation of an interface device in accordance with one embodiment of the invention wherein the interface device is used as primary interface device;

FIG. 5 is a schematic diagram of an interface device in accordance with one embodiment of the invention wherein the interface device is used as a primary interface device without a keyboard and/or mouse;

FIG. 5A is a schematic diagram of an interface device in accordance with one embodiment of the invention wherein the interface includes a force/pressure sensor;

FIG. 6 is a schematic diagram of the operation of an interface device in accordance with one embodiment of the invention;

FIG. 7 is a schematic diagram of a menu system in accordance with the invention;

FIG. 8 is a representative flow chart of a menu module for prompting a user to select the type of input device and axes;

FIG. 9 is a representative flow chart of a menu module for prompting a user to select input sensitivity;

FIG. 10 is a representative flow chart of a menu module for prompting a user to input axis parameters;

FIG. 11 is a representative flow chart of a menu module for prompting a user to select tolerance values for an axis;

FIG. 12 is a schematic diagram showing tolerance sectors for two axes for both translational and rotational movement;

FIGS. 13A, 13B and 13C are representative flow charts of menu modules for prompting a user to configure specific input devices;

FIG. 14 is a representative flow chart of a menu module for actively connecting a configured interface device to a computer;

FIG. 15 is a representative flow chart of a menu module for prompting a user to test configuration settings of an input device; and,

FIG. 16 is a schematic diagram of modules of interface device in accordance with one embodiment.

### DETAILED DESCRIPTION

With reference to the Figures, the present invention relates to a portable electronic interface device (ID) 10 enabling the operative connection of a single or multi-axis movement sensing or tactile force system 12 into recognizable input commands to a computer 14 to control that computer or to otherwise enable a user 16 to interact with the computer and its software.

In particular, in a preferred embodiment, the system enables a user to play video games on the computer using non-conventional input namely movement or force sensors that may be configured to a variety of common or known objects or rehabilitation objections. Representative examples of sensor systems are explained in greater detail below.

In a preferred embodiment, the input system is a magnetic motion sensor (MMS) system 12 the MMS including a magnetic field generator, a motion sensor 12a and hardware/software for determining the position and orientation (typically six degrees of freedom including three position and three orientation signals) of the motion sensor within a magnetic field. For the purposes of this description, while the system can utilize a variety of input systems to provide almost any form of input to a computer, this description is written in the context of video game input and an MMS system.

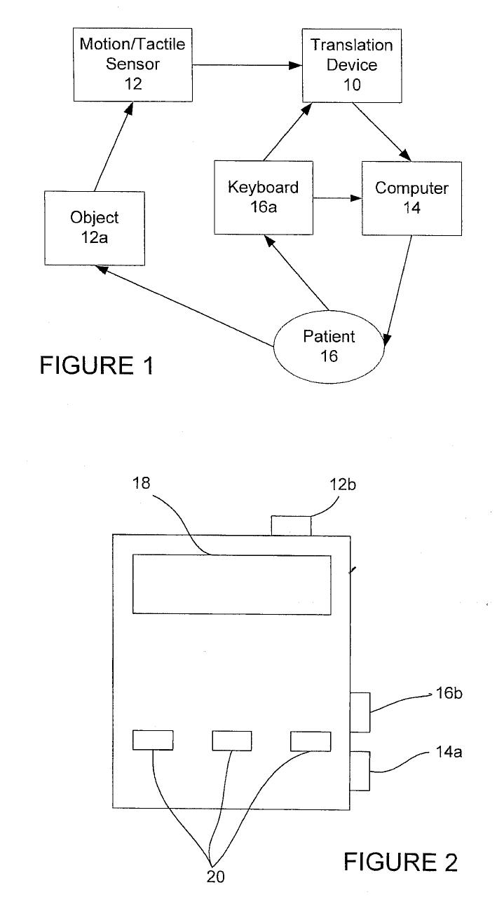

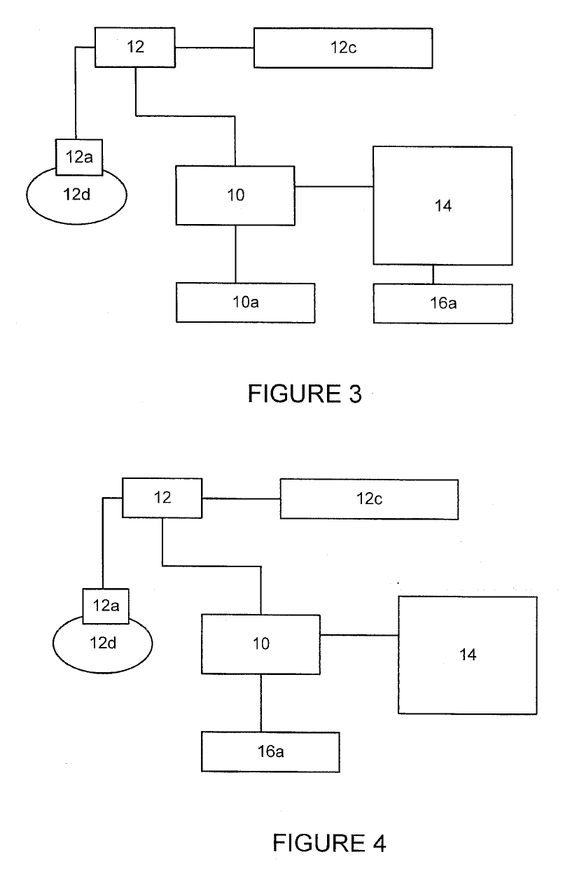

The ID 10 is a configurable and embedded portable electronic interface that receives measured and calibrated signals from an MMS, independently translates one or a combination of the position and orientation signals from the MMS into a digital output signal for input to a computer. The digital output is equivalent to a standard input device 16a such as a peripheral mouse, analog/digital joystick, analog/digital game pad or keys from a standard PC computer keyboard. More specifically, the ID provides the ability to precisely map in real-time the MMS motion to that of a mouse, analog/digital joystick, analog/digital game pad, keys from a standard PC computer keyboard or any combination thereof as shown schematically in FIGS. 1 and 2.

With reference to FIGS. 1 and 2, the ID 10 preferably includes an interface 12b for connection to an MMS input system 12, an interface 16b for connection to a keyboard 16a, a USB interface 14a for connection to a computer or console 14, a display 18 and appropriate user control buttons 20.

Preferably, the ID is compatible with the most common video game controllers input devices (1-, 2- and 3-dimensional), and enables a user operating the MMS to play commercially available video games. That is, by attaching the motion sensor of the MMS to a wide-range of objects or a body part and by moving that object or body part, the user can effectively interface with the computer by movements and/or body contact forces not normally enabled by a standard input device.

The electronic interface is not restricted to PC computers but can also be connected to and function on video game consoles, such as, Microsoft XBOX, Sony Playstation2, Nintendo Game Cube, etc.) and enable a player to play various commercially available video games on these platforms.

The ID is particularly beneficial as a system and method to assist persons with movement disorders, restrictions, limitations or difficulties to engage in effective rehabilitation therapies. Such disorders may include but are not restricted to:
a) Neurological disorders including stroke, cerebral palsy, Parkinson, s disease, traumatic brain injuries, spinal cord injuries, Multiple Sclerosis and diabetes, and,
b) Muscular-skeletal disorders including arthritis, amputees, carpel tunnel syndrome and traumatic and sport injuries.

The interface device can also be used for ergonomic analysis and training of many normal or hazardous working skills or tasks and situations.

A preferred embodiment of the system is described.

### Pulsed Motion Magnetic Motion Sensors

In a preferred embodiment, the system uses a miniaturized six degree-of-freedom position and orientation tracking sensor as the mechanical interface and for providing input to the system. Pulsed DC magnetic motion sensors are used as input signals to the MMS and permit the system to accurately track in real time (with an unrestricted range of motion) the spatial location and orientation of an object or body segment within the magnetic field of the MMS.

An example of commercially available DC magnetic motion sensors and accompanying digital electronic interface and electromagnet is the miniBird™ system from Ascension Technologies (website: www.ascension-tech.com). These sensors are both miniature and reliable and accurately measure the instantaneous position and orientation of the sensor with six degrees of freedom at a frequency of up to 100 Hz. The DC magnetic motion sensors can measure linear position in 3-dimensional space (x, y, and z) and rotations also in 3 directions (roll, yaw, pitch). DC magnetic motion sensors from Ascension Technologies are available in different sizes including the miniBird having dimensions of 18 mm×8 mm×8 mm for the Model 800 and 10 mm×5 mm×5 mm for the Model 500. The weight of the sensors is less than 21 grams. Pulsed DC magnetic fields avoid blocking problems and yield-reduced distortion from nearby conductive metals. Signals pass through the human body without attenuation. Although slightly larger, the Polhemus Patriot™ is another readily available commercial DC magnetic sensor for six-degree-of-freedom motion tracking up to 60 measurements a second (website: www.pollhemus.com). The DC magnetic motion sensors are packaged with necessary digital electronic interfaces and electromagnets for generating the reference magnetic field.

The basic principle of operation of the ID is to transform the widest range of objects into a functional 2D or 3D video game controller, in particular a mouse, joystick, game pad or cursor keys of a keyboard. The ID and system allows the user to easily select the type (linear/angular) and direction (x, y, z axis) of object motion to be used in playing the video game. Virtually any object or body part can be chosen, as appropriate, and transformed into a video game input device. A user can choose the type and direction of motion to represent how the object would be handled and manipulated in real life functions. Objects that require one or two hands to be manipulated can be selected.

In operation, it is important that the user playing a video game is competitive in playing the video game; otherwise the user will likely become frustrated with the system and quickly lose interest.

For this purpose, the ID system has configurable manipulation ID algorithms to augment limited movements of a user in a number of ways. Thus, besides the ability to select among virtually any object and the type and direction of the object motion or levels of contact forces to use for game controls, the system can scale and amplify movement signals for those users who leave small or very small movements. In addition, the ID algorithms can smooth and filter signals from tremor and jerky movements and a variety of other important movement transformations or contact force.

Given that the connected PC computer or game console just sees the output signal of the ID as a standard peripheral USB input device (configurable to either a mouse, joystick, game pad, keyboard keys or combination thereof), any special settings within any gaming environment also can be applied in conjunction with a variety of options designed to make the game playable for patients with severely limited and restricted movement. This allows a great flexibility of options that can be individualized for each user's needs.

The premise of this rehabilitation therapy is to encourage active movements of the fingers, hands and arms while manipulating real objects in real ways. Many repetitions of these goal-directed functional movements performed in a random manner are critical to recovery. By making practice fun and exciting, coupling exercises to video games, more practice time and volume can be achieved.

The design of the system allows for a working PS/2 keyboard (or other keyboard such as USB) to be hooked up simultaneously as the DC magnetic motion sensor. This allows for the unit to be a complete keyboard replacement if necessary. The Microsoft Windows OS (or other operating system with HID compatibility) will treat the unit as a plug-n-play device to greatly reduce installation difficultly. The unit is universally compatible with Windows (98/2000/XP), Apple Macintosh, various video games consoles that support USB or have controllers based on USB design, and Linux Machines with a 2.4 kernel or greater, or 2.2 kernels with USB extensions.

### Software Description

The system uses several means of control and configuration in which to assist both the user (patient) and rehabilitation facilitator. The controller interface integrates with an operating system or console that supports a generic HID interface for mouse, joystick, game pad or keyboard. Any system that uses this protocol will therefore support the controller without need for specialized device drivers to be constructed. Specialized drivers can be used to enhance interaction between the interface (ID) and target computer console. In other embodiments, the data and configuration output from the interface can directly input to a console via the proprietary interface of that system.

### Device Configuration

The output of the interface device dynamically selects the combinations of keyboard, mouse, and joystick outputs. For each function there are test modes to ensure that there is proper communication between the device and the computer or console. Device configuration can also be accomplished via specialized software on the console depending on the preference of the facilitator.

### Movement Configuration

Position and tilt sensors, for example the six-degree-of-freedom DC magnetic motion-sensor, detects and relays position on an X, Y, and Z-axis along with orientation in pitch, yaw, and roll directions. The facilitator, or therapist, is able to change the parameters of the device via the control console on the ID or via an external configuration software interface. Thus various configurable combinations of linear and/or angular motion about any axis can be used as input to the device to be translated into the desired game controller (mouse, joystick game pad and/or keyboard). The facilitator can adjust a movement range with a central point. The central point can be adjusted to allow for an area in which all movement, relative or otherwise, is nullified, similar to a stop-band filter. Utilizing this method, any type of movement along various axes can be easily controlled by the patient.

### Scale Configuration

The scale configuration system allows for the range of movement given by the input system to be scaled fitting the range of the patient undergoing a given exercise. Each user/patient has different ranges of capable motion that can be addressed by this scaling ability. The scale configuration system allows the facilitator to adjust the parameters of the motion (or contact forces) to reflect the range of motion (and/or forces) required by the video game being played on the computer or console. Each axis is separately configurable and scalable via the console or via an external configuration software interface.

### Diagnostic Inputs/Outputs

The system enables the user/facilitator to test output and input signals via the diagnostic console. This allows simulation of the output to verify that it is compatible with the video game(s) being used. As all configurations are dynamic, meaning changeable at any time, it is important that the ability to verify the settings of the system exists. The diagnostic console is able to display the current settings used, as well as view and test input directly from the input system in a built in diagnostic terminal.

### Input/Output Assists

The device is also capable of providing secondary output assistance. Within various genies of games, there is a need for the user to hold down an acceleration key in racing games, or a fire button for first-person or arcade-style games. Recognizing this need, the device allows for 8 output buttons/keys to be pressed at independent, dynamically configurable intervals.

### Specific System Functionality and Variations

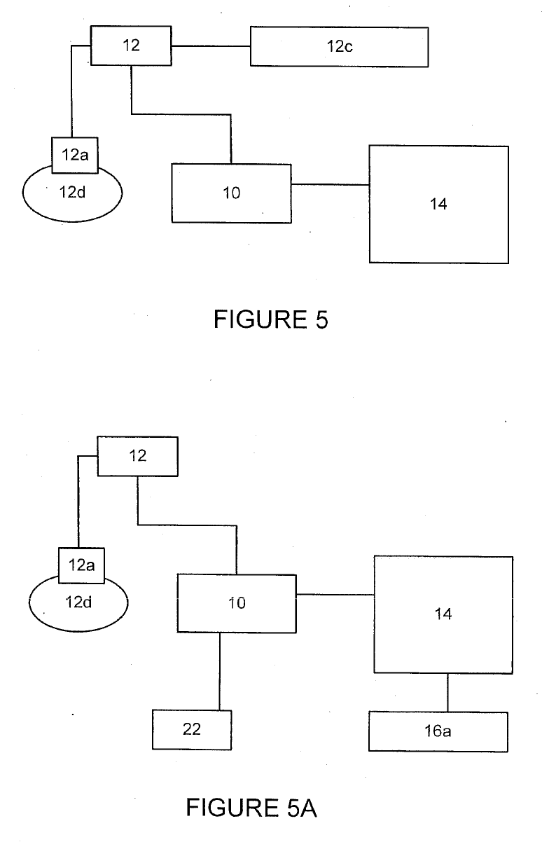

Within the client or patient environment, the magnetic field generator 12c is placed within the area that the patient is performing the exercises as shown in FIGS. 3, 4 and 5. This is usually done within easy view of a computer monitor or other viewable device, such as a television in cases of a video game console. The magnetic sensor is then placed on an object 12d that is tasked for manipulation by the patient in the context of rehabilitation or other reasons. The MMS sensor control box 12 is attached to the ID. In turn the ID 10 is attached to a personal computer 14, video game console or any input device that will accept the signals as desired.

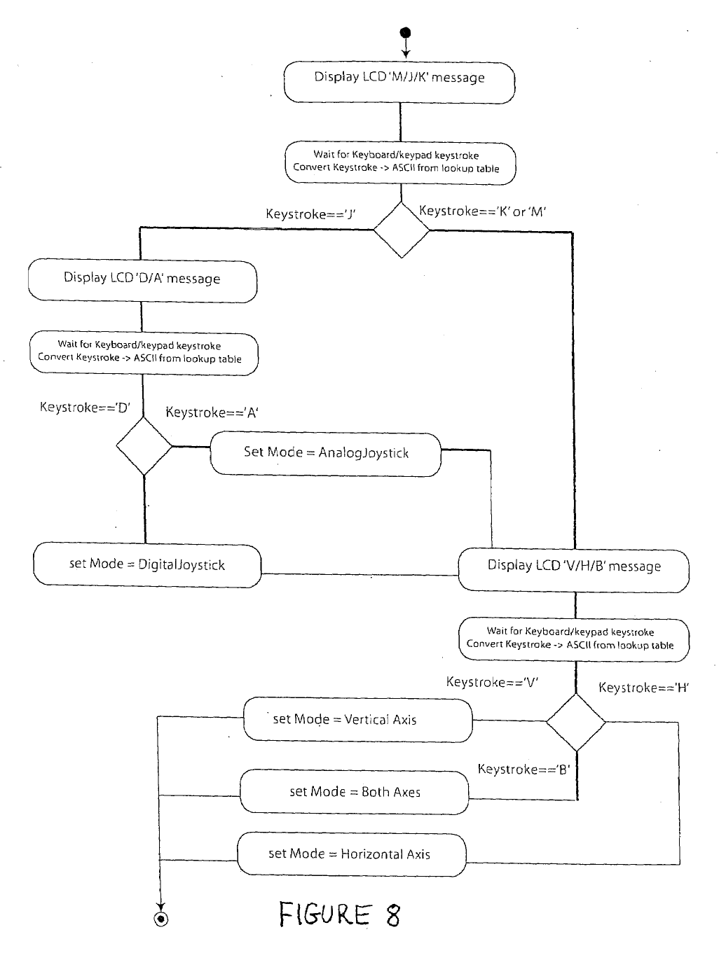

With reference to FIG. 6, a schematic diagram of the generalized operation of the ID is described. After connection of the ID to the MMS and a computer/video game, the MMS input and output to the computer/video game are initialized. If the ID processor detects a problem during initialization, an error message is displayed.

If no initialization problems are detected, the user menu is displayed. The input to the ID is configured by providing the user with a choice of other input devices such as a joystick, mouse and/or keyboard and mapping the input signals of the standard input device (ie vertical movement, horizontal movement, keystrokes, etc) to various movements of the MMS. This mapping may include configuring individual or combinations of movements. This configuration information is sent to an output relay in order to relay the correct information according to the input device specifications.

Thereafter, the ID will prompt the user to enter a combination of middle point and/or end points to determine relative change in position as detected by the motion tracking sensor for each defined input movement. In this step, various combinations of spatial points are set by the user by defining the anticipated maximum positions and middle point positions that the user will move through when playing the game for each input movement. This step is performed in order that changes in position correspond to appropriate changes in joystick/mouse/keyboard output and to set the boundaries of movement.

During use, the motion tracking sensors constantly sends information on an absolute position. The ID, based on the type of output, determines if the output signal should send the equivalent of an X,Y delta or set the X, Y coordinates based on the change of position from the old position to the current one. If during use, the ID determines that user movement (or force) is “in bounds”, the appropriate output signal is delivered to the computer interface. Otherwise, if the ID determines that movement is repeatedly “out of bounds”, an appropriate warning is provided to the user to encourage movement “in bounds” which may also display the user menu in the event that the ID requires reconfiguration.

In various embodiments, configuration of the ID may be controlled by drivers located either on the ID or on the gaming computer. That is, the configuration routines may be physically located on the gaming computer wherein the appropriate displays and/or prompts to complete configuration are displayed on the gaming system display. In this embodiment, the configuration routines would query the ID to obtain the necessary configuration data from the ID to effect configuration as described above.

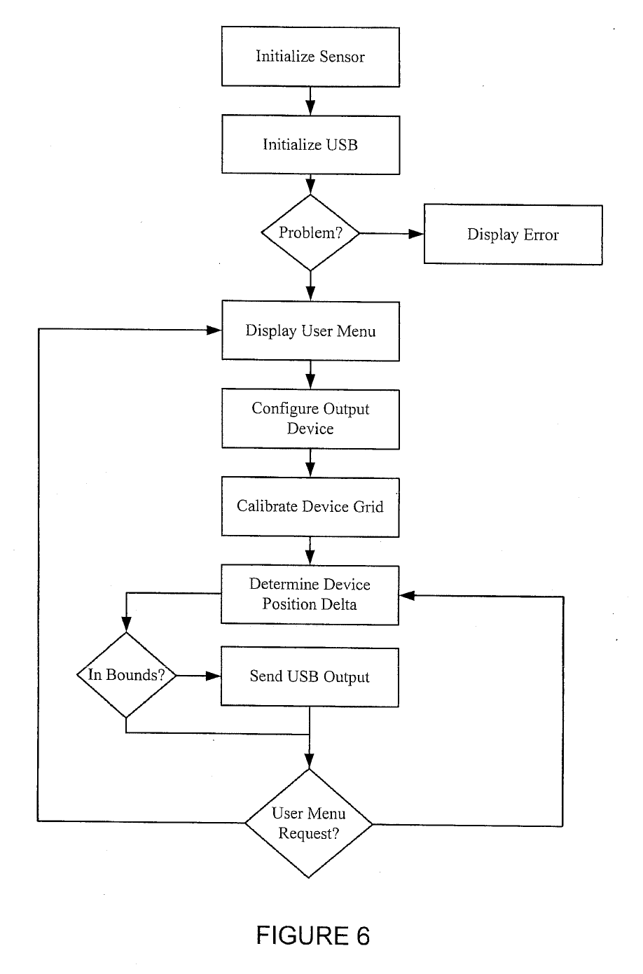

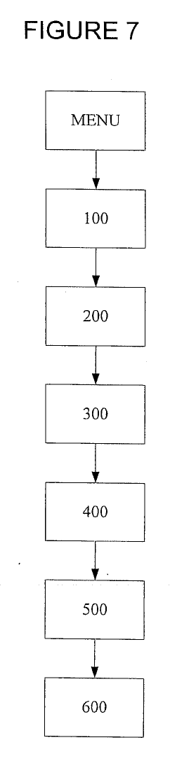

Specific embodiments of the system are shown in FIGS. 3, 4 and 5 where the ID 10 is used as a secondary input device (FIG. 3), as the primary input device to control computing or gaming (FIG. 4) and where the ID 10 is used without any keyboard input (FIG. 5).

### Sensor Types

In accordance with various embodiments of the invention, the interface device is adapted for use with different types of motion and biological sensors to provide appropriate input signals including position and tilt sensors and force sensors. Position sensors measure position to track the actual motion of an object while it is being handled or manipulated by measuring or tracking lineal displacements of an object whereas tilt sensors track or measure angular displacement of an object.

Force sensors measure contact forces between fingers and objects that are being handled or manipulated.

There are many choices of sensor technologies which provide high accuracy and wide angular range measurements.

Examples of Position sensors which can be used as input signals include:
i) Pulsed DC magnetic sensors (miniBIRD system, Ascension Technologies)
ii) Resistive sensors. Linear potentiometers are the best-known and most frequently used resistive sensors. Resistive sensors use a moving contact sliding against a fixed resistive element to generate changes in resistance. When connected to a DC source as a voltage divider, resistive sensors produce a proportional voltage output. There are many types and sources of resistive sensors which can be found at almost any electronic store or website. Agnetoresistive sensors are a contact-free variation on the linear potentiometer that use moving magnets, thus eliminating wear problems.
iii) Time-of-flight sensors send out a wave signal (for example sound) toward a moving target and measure the time it takes to receive a reflection off the target. Ultrasonic time-of-flight sensors-depend on timing the reflection of sound and light, respectively. Both are contact-free.
iv) Laser Ring sensors are an optical rotation sensor comprising a light amplification stimulated emission ring laser formed in a solid medium and that is provided with phase modulator devices which produce push-pull phase modulation of counterpropagating optical waves in the ring thereby enhancing bidirectional lasing in single longitudinal modes in each direction. Using multiple sensors coupled to the interface device can yield three-dimensional positional data.

Examples of Tilt sensors which can be used as input signals include:
i) Pulsed DC magnetic sensors (miniBIRD system, Ascension Technologies)
ii) Accelerometers that measure static earth's gravity field. Unlike Piezoelectric or Piezofilm sensors which only respond to dynamic acceleration, there are a class of accelerometers (Analog Devises Inc, USA) that provide a DC response by sensing static acceleration due to earth's gravity.
ii) Electrolytic tilt sensors
iv) 3DM-DH a micro miniature 3-axis tilt sensor which uses orthogonal arrays of agnetometers and accelerometers (Microstrain, Inc. USA).

Examples of Force Sensors providing high accuracy and a wide range measurements of finger to object contact forces during handling and manipulation tasks and exercise include pizo-resistivo sensors (force sensitive resistor). These sensors are thin (less than 0.2 mm) and flexible tactile force sensor. Single sensors are available in a wide range of shapes, sizes (for example ½ to 1 cm). These sensors are sensitive and capable of measuring finger-object contact forces ranging from 0.1 PSI to 30 PSI (Vista Medical, Canada and Tekscan, Inc. USA.). These sensors can be attached to, and thus instrument, objects utensils, tools and fingers and hands used in basic activities of daily living, instrumental activities of daily living and work or leisure activities. An embodiment of the invention utilizing a force sensor 22 as shown in FIG. 5A where the force sensor is operatively connected to the interface device 10.

### Menu System

In preferred embodiments, the ID includes a menu and underlying software that allows for rapid configuration of the system.

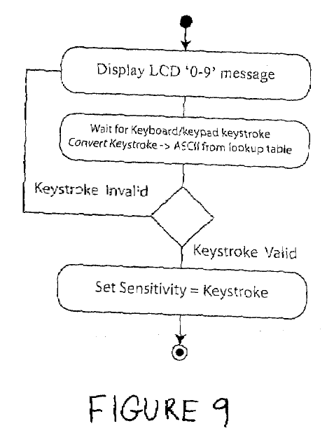

For example, as shown in FIG. 7, the user in configuring the system for video game play may step through a number of menus to enable an appropriate input device to be configured to the system. Various menu modules are generically referenced as boxes 100, 200, 300, 400, 500 and 600. As will be explained in greater detail below, a specific order of execution of various menu options is not necessarily required and will depend on the type of device being configured, the game being played and the movements that are desired to provide the game input.

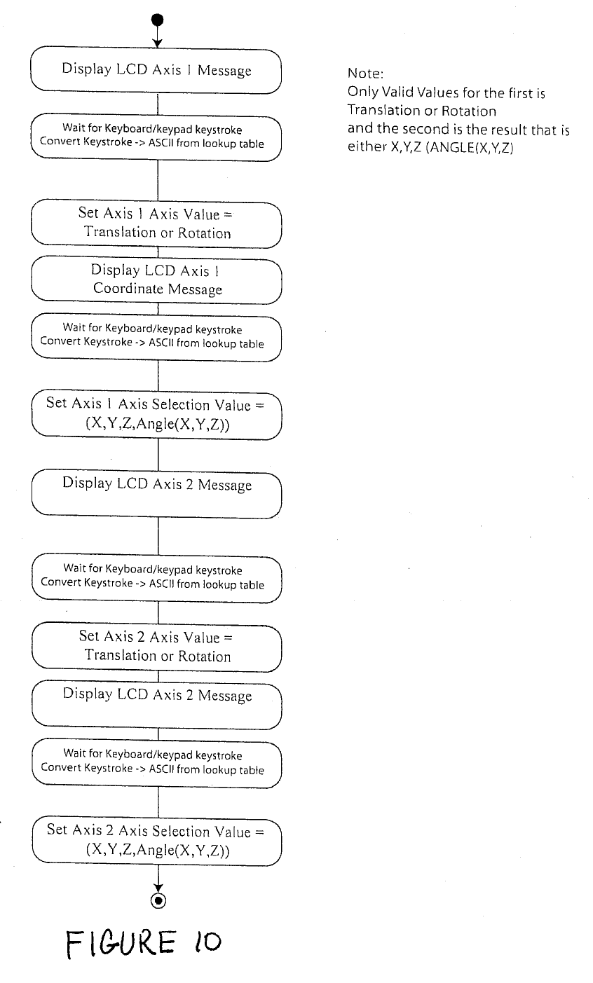

Initially, a user may be prompted to select the type of input device to be configured as shown in a typical flowchart sequence (FIG. 8). As shown in FIG. 8, a user may be prompted to select between various modes such as “analog joystick”, “digital joystick”, “keyboard mode” or “mouse mode” by appropriate menu prompts and keystrokes as well as to set modes as to which of vertical, horizontal or both axes may be utilized.

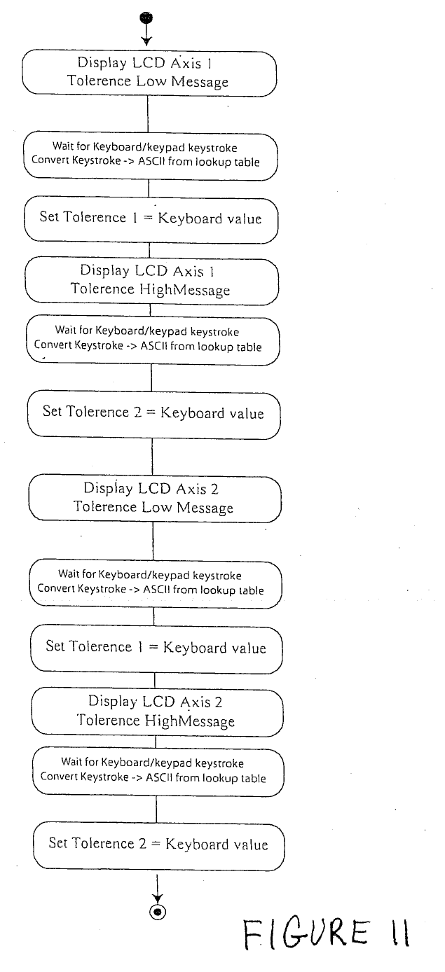

As shown in FIG. 9, the user may be prompted to select the relative sensitivity of movements within each axis of movement by appropriate menu prompts and keystrokes.

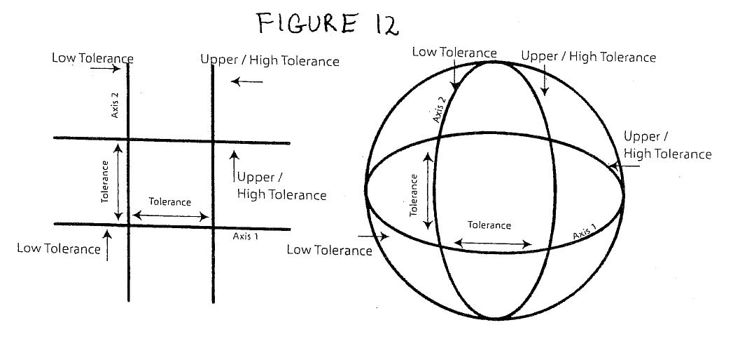

As shown in FIG. 10, the user may be prompted to select and enter values representing appropriate movement parameters for each axis of movement. For example, these may include setting values for whether movement is translational or rotational, initial coordinates and axis selection values including angular positions. Such values are set for each relevant axis.

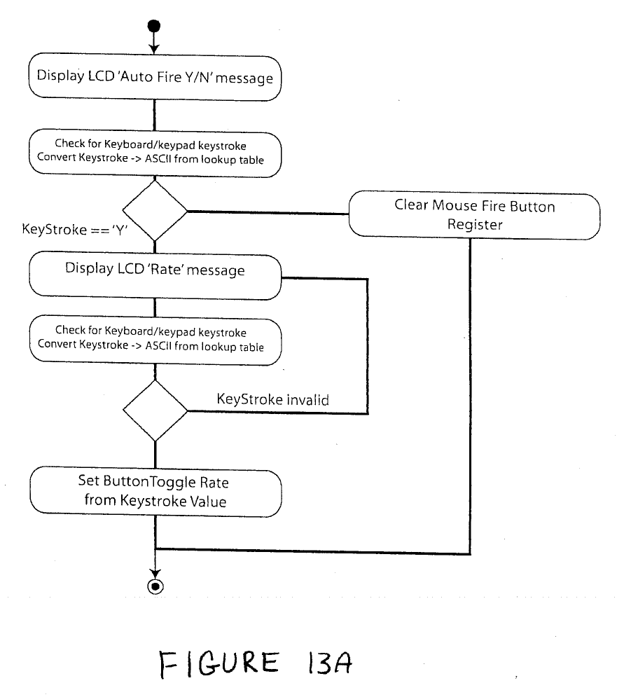

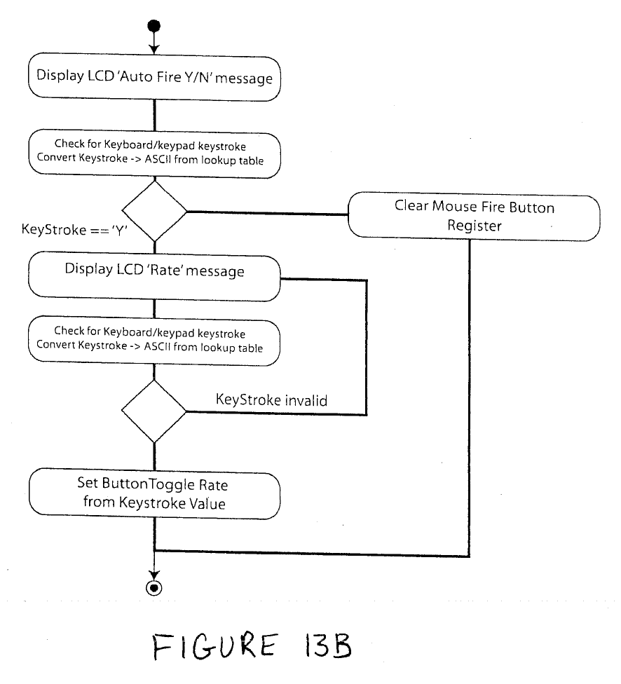

As shown in FIGS. 11 and 12, the user may be prompted to select and enter threshold or tolerance values for each axis for both translational and rotational movements. A representative grid for both translational and rotational movements is shown in FIG. 12 showing various sectors where the threshold or tolerance values may be set to define such sectors. By setting threshold or tolerance values, the desired movements from the input system may be further refined to reflect known movements from conventional input systems into computer games.

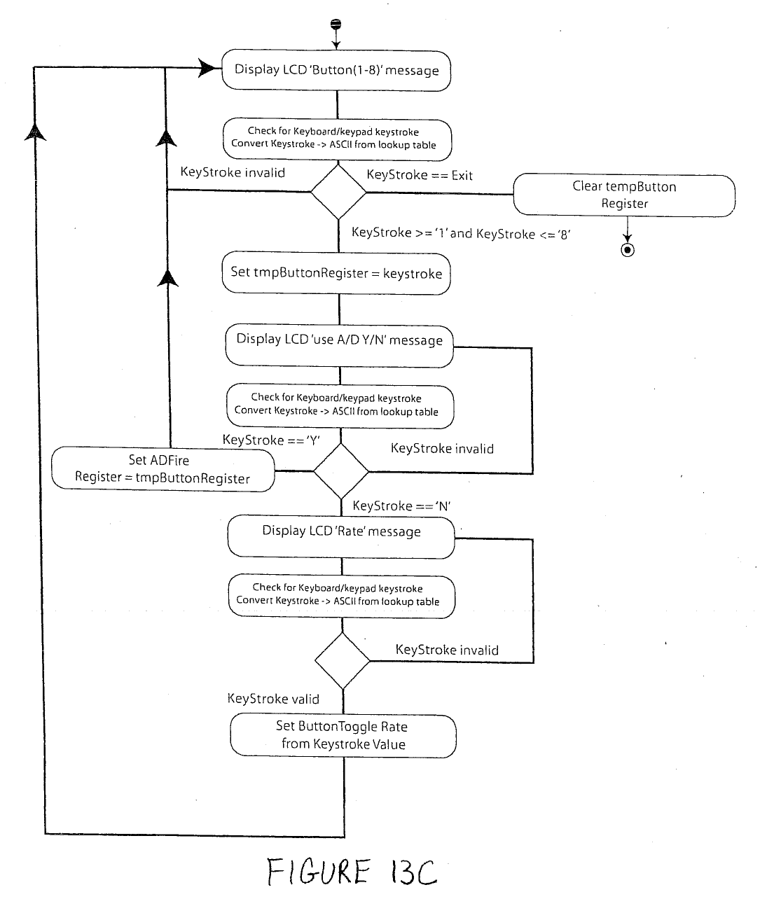

As shown in FIGS. 13A, 13B and 13C, the user may be prompted to select and enter values to further refine the input from various input systems including setting pressure sensitivity for external buttons (A/D sensitivity), rates, “auto-fire” and other appropriate values to assist mapping movements as useful computer input.

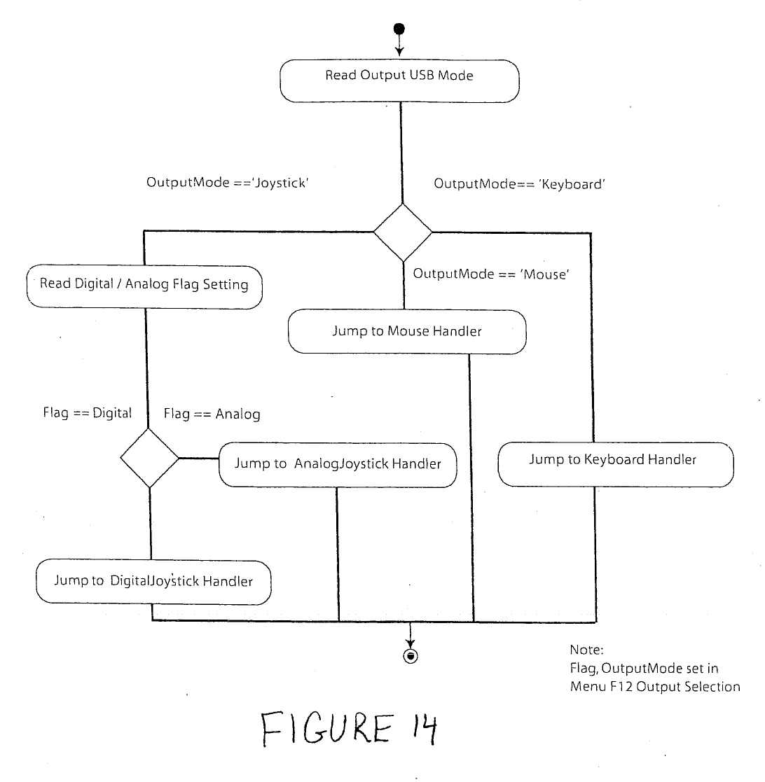

After setting all appropriate input parameters as described above, the ID may be made active in accordance with a representative procedure as shown in FIG. 14. In other words, upon completion of the input device configuration, the ID with its configuration settings may be operatively connected to the computer game through a menu option.

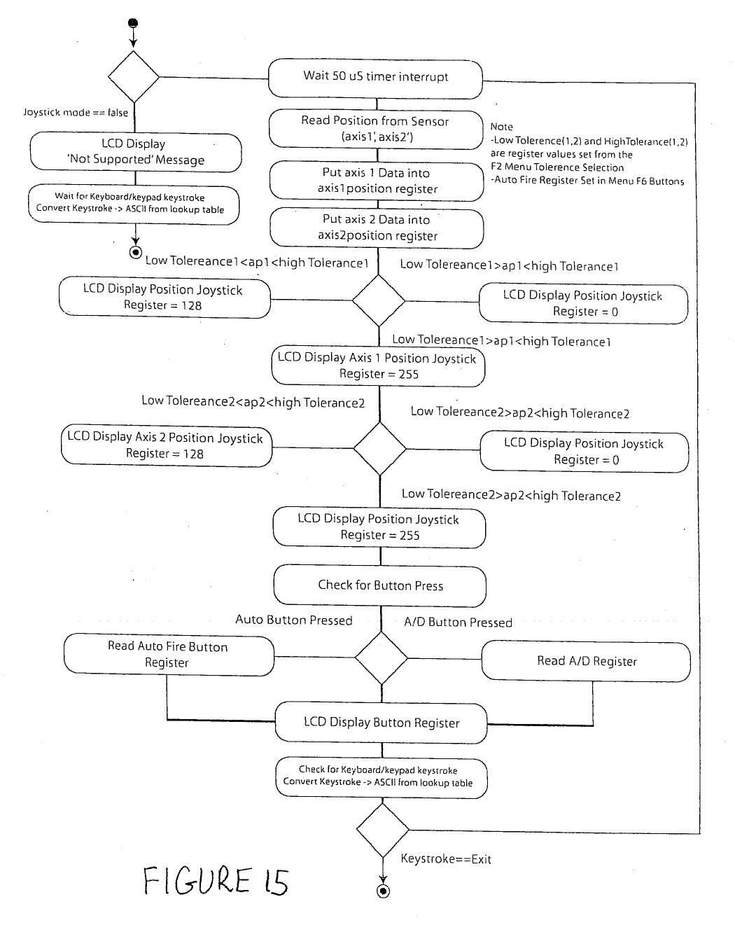

The system may further include appropriate diagnostic and test modes to enable a user to verify that configured input movements are as desired as shown in FIG. 15. Within this figure, a representative procedure for verifying positions, tolerances or thresholds, button activation and other parameters are shown.

The menu system may be accessed through specific keystrokes such as F1-F12 keys on a keyboard, touch screen, mouse input etc. as known to those skilled in the art.

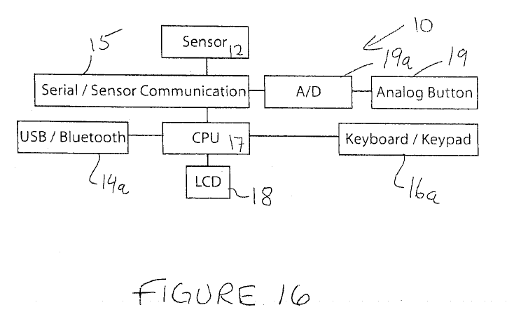

A component diagram (FIG. 16) shows representative modules of the ID 10 in accordance with the invention. The ID includes a CPU 17 operatively connected to an LCD display 18, a USB/Bluetooth port 14a, keyboard 16a and serial/sensor communication module 15. The serial/sensor communication module 15 is operatively connected to a sensor 12 and analog input system 19 through an A/D interface 19a.

The above-described embodiments of the present invention are intended to be examples only. Alterations, modifications and variations may be effected to the particular embodiments by those of skill in the art without departing from the scope of the invention, which is defined solely by the claims appended hereto.

1. An interface device for operative connection between a computer and a motion input system comprising:
a first interface for operative connection to the motion input system and a second interface for operative connection to the computer; and
a processor operatively connected to the first and second interfaces for receiving input signals from the motion input system and enabling configuration of the input signals to output signals for input to the computer for controlling the computer.

2. An interface device as in claim 1 wherein the motion input system is a one, two or three dimensional motion input system.

3. An interface device as in claim 1 wherein the motion input system is a tactile force input system.

4. An interface device as in claim 1 wherein the motion input system is combination of a tactile force and a one, two or three dimensional input system.

5. An interface device as in claim 1 wherein the processor includes means for dynamically selecting any one of or a combination of keyboard, mouse and joystick equivalent outputs.

6. An interface device as in claim 1 wherein the processor includes means for testing communication between the interface device and the computer.

7. An interface device as in claim 1 wherein the processor includes means for receiving input from a user to adjust the linear and angular motion of a sensor of the input system to a defined range of motion for input to the computer.

8. An interface device as in claim 7 wherein the processor includes means for selectively excluding selected linear or angular motion of a sensor based on the measured movement of the sensor by a user.

9. An interface device as in claim 7 wherein the processor includes means for selectively smoothing input signals from the sensor based on the measured movement of the sensor.

10. An interface device as in claim 7 wherein the processor includes means for selectively amplifying input signals from the sensor based on the measured movement of the sensor.

11. An interface device as in claim 1 wherein the processor includes means for testing input and output signals for simulating output signals to verify compatibility with the computer's software.

12. An interface device as in claim 1 wherein the processor includes means for providing output signals to a USB port.

13. An interface device as in claim 1 further comprising a keyboard input interface operatively connected to the processor for providing keyboard input to the processor.

14. An interface device as in claim 1 further comprising a keypad input interface operatively connected to the processor for providing keypad input to the processor.

15. An interface device as in claim 1 further comprising a display operatively connected to the processor for displaying graphic output to the user.

16. An interface device as in claim 1 wherein the processor includes firmware enabling a menu system for configuring the motion input system.

17. An interface as in claim 16 wherein the menu system prompts a user to select a class of input device.

18. An interface device as in claim 16 wherein the menu system prompts a user to select input system sensitivity.

19. An interface device as in claim 16 wherein the menu system prompts a user to enter values representing movement parameters for each axis of movement.

20. An interface device as in claim 16 wherein the menu system prompts a user to enter threshold or tolerance values for each axis for both translational and rotational movements.

21. An interface device as in claim 20 wherein the threshold or tolerance values are set to define tolerance sectors.

22. An interface device as in claim 16 wherein the menu system prompts a user to access a test mode for verifying input system configuration.

23. An interface device as in claim 1 wherein the interface device is plug and play.

24. A method of providing video gaming functionality to patients having compromised motion comprising the steps of:
a. operatively connecting the interface device of claim 1 between a motion input system and a computer;
b. operatively connecting an exercise object capable of manipulation by the patient to a motion sensor of the motion input system, and,
c. configuring the interface device such that movement of the motion input system results in appropriate input to a video game on the computer.

25. A method as ill claim 24 wherein the step of configuring the interface includes any one of or a combination of:
a. selectively excluding selected linear or angular motion of a sensor based on the measured movement of the sensor by a user;
b. selectively smoothing input signals from the sensor based on the measured movement of the sensor by a user; and,
c. selectively amplifying input signals from the sensor based on the measured movement of the sensor by a user.

26. A rehabilitation system comprising:
a. an interface device as in claim 1;
b. a motion input system including at least one sensor, the motion input system for operative connection to the interface device and the sensor for operative connection to at least one rehabilitation object;
c. a computer system for operative connection to the interface device, the computer system having video games;
wherein a user may play a video game on the computer system by manipulation of the rehabilitation object.

27. A rehabilitation system as in claim 26 wherein the at least one sensor is selected from any one of or a combination of a pulsed DC magnetic sensor, a resistive sensor, a time-of-flight sensor, a laser ring sensor, a static accelerometer, an electrolytic tilt sensor, a micro miniature 3-axis tilt sensor±sensor, and a peizo-resistivo sensor.
